(19) United States
(12) Patent Application Publication
(10) Pub. No.: US 2026/0325882 A1
(43) Pub. Date: Oct. 22, 2026

(54) SYSTEMS AND METHOD OF HISTOTRIPSY BLADDER THERAPY

(71) Applicant: HISTOSONICS, INC., Ann Arbor, MI (US)

(72) Inventor: Joshua Stopek, Minneapolis, MN (US)

(21) Appl. No.: 19/551,297

(22) Filed: Feb. 26, 2026

Related U.S. Application Data
(60) Provisional application No. 63/763,828, filed on Feb. 26, 2025.

(51) Int. Cl. A61N 7/00; A61B 34/30

(52) U.S. Cl. CPC A61N 7/00; A61B 34/30; A61B 2034/301; A61B 2034/303; A61N 2007/0039; A61N 2007/0043

(57) ABSTRACT

A system and method for applying histotripsy therapy to a bladder including an acoustic coupling assembly configured for placement on the patient, a fluid source and configured to supply an acoustic coupling fluid acoustic coupling assembly, and a therapy transducer configured to emit an ultrasound signal and generate a bubble cloud at a focal point of the therapy transducer, wherein the therapy transducer is configured to acoustically couple to tissues of the bladder of the patient via the acoustic coupling assembly and fluid within the bladder of the patient.

### FIELD

This disclosure is directed to systems and methods of planning and conducting therapy of bladder conditions using local high intensity therapeutic ultrasound (HITU).

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, histotripsy realm; it is necessary to deliver acoustic energy in the form of high amplitude acoustic pulses with low duty cycle.

Compared with conventional focused ultrasound technologies, histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) histotripsy produces lesions in a controlled and precise manner. It is important to emphasize that unlike thermal ablative technologies such as microwave, radiofrequency, high-intensity focused ultrasound (HIFU), cryogenic, or radiation, histotripsy relies on the mechanical action of cavitation for tissue destruction and not on heat, cold or ionizing energy.

### BRIEF DESCRIPTION OF THE DRAWINGS

The features of the disclosure are set forth with particularity in the claims that follow. A better understanding of the features and advantages of the disclosure will be obtained by reference to the following detailed description that sets forth illustrative embodiments, in which the principles of the disclosure are utilized, and the accompanying drawings of which:

FIG. 1A is a perspective view of a histotripsy system in accordance with the disclosure.

FIG. 1B depicts a therapy transducer and imaging transducer in accordance with the disclosure.

FIG. 2 depicts a histotripsy system deployed to treat a patient in accordance with the disclosure.

FIG. 3 is a plot of a pressure wave during application of a histotripsy ultrasound pulse in accordance with the disclosure.

FIG. 4A is a representation of a bladder with tumors.

FIG. 4B shows a resectoscope inserted into the a bladder.

FIG. 5 depicts a method for treating bladder tumors using histotripsy.

FIG. 6 depicts an example of one image from a CT scan of a patient's bladder depicting tumors manifest around the bladder.

FIG. 7 is an example of a bladder with treatment volumes overlapping in an ellipsoid shape.

FIG. 8 is a flowchart depicting a method of treating a tumor with histotripsy.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing, and ablation. Furthermore, due to tissue selective properties, histotripsy may be used to create a cytoskeleton that allows for subsequent tissue regeneration either de novo or through the application of stem cells and other adjuvants. Histotripsy can also be used to cause the release of delivered agents such as chemotherapy and immunotherapy by locally causing the release of these agents by the application of acoustic energy to the targets. As will be described below, the acoustic cavitation system may include various sub-systems, including a cart, therapy, integrated imaging, robotics, coupling and software subsystems. The acoustic cavitation system also may comprise various other components, ancillaries, and accessories, including but not limited to computers, processors, memory, software, applications, cables, connectors, networking devices, power supplies, displays, drawers/storage, doors, wheels, and various simulation and training tools, etc. All systems, methods and means of creating/controlling/delivering histotripsy are considered to be a part of this disclosure.

### The System

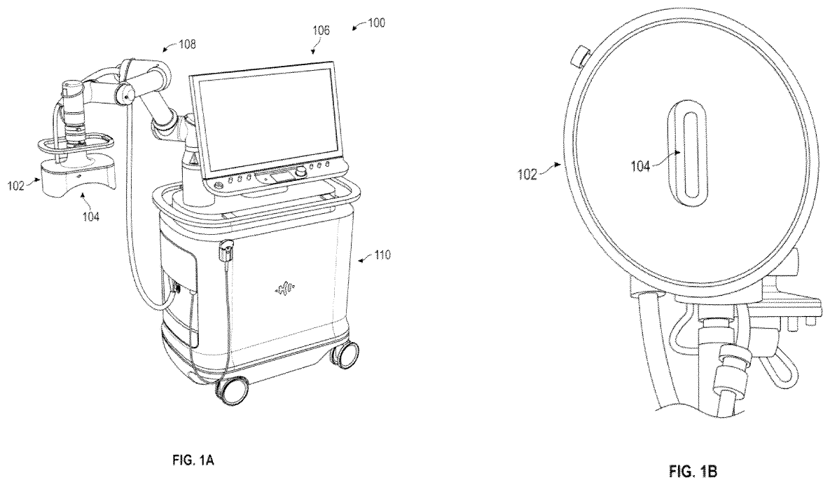

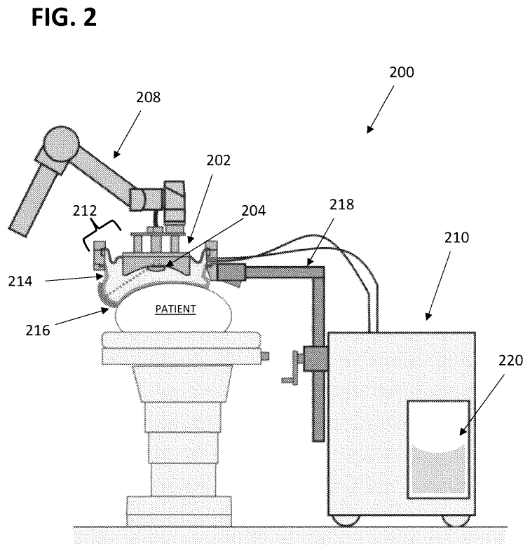

FIG. 1A depicts a histotripsy system 100 in accordance with the disclosure. The histotripsy system 100 includes a therapy transducer 102, an imaging system 104, a display and control panel 106, a robotic positioning arm 108, and a cart 110. The system can further include an ultrasound coupling interface and a source of coupling medium (FIG. 2).

FIG. 1B is a bottom view of the therapy transducer 102 and the imaging system 104. As shown, the imaging system 104 can be positioned in the center of the therapy transducer 102. However, other embodiments can include the imaging system 104 positioned in other locations within the therapy transducer 102, or even directly integrated into the therapy transducer 102. In some embodiments, the imaging system is configured to produce real-time imaging at a focal point of the therapy transducer 102. The system also allows for multiple imaging transducers 104 to be located within the therapy transducer 102 to provide multiple views of the target tissue simultaneously and to integrate these images into a single 3-D image.

The histotripsy system 100 may comprise, and the cart 110 may enclose, one or more of various sub-systems, including a therapy sub-system that can create, apply, focus and deliver acoustic cavitation/histotripsy through one or more therapy transducers 102, an integrated imaging sub-system (or connectivity thereto) allowing real-time visualization and display of the treatment site and histotripsy effect through-out the procedure (e.g., via, a robotics positioning sub-system to mechanically and/or electronically steer the therapy transducer) and further enabled to connect/support or interact with a coupling sub-system to allow acoustic coupling between the therapy transducer 102 and the patient, and software to communicate, control and interface with the system and computer-based control systems (and other external systems) and various other components, ancillaries and accessories, including one or more user interfaces and displays, and related guided work-flows, all working in part or together. The histotripsy system 100 may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described in greater detail below, the cart 110 may be configured and arranged to be used in a radiology environment and in some cases in concert with imaging (e.g., computed tomography (CT), cone beam CT and/or magnetic resonance imaging (MRI) scanning). In other embodiments, the cart 110 may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The cart 110 may also work with a patient surface (e.g., table or bed) to allow the patient to be presented and repositioned in a variety of positions, angles and orientations, including allowing changes to such to be made pre, peri and post-procedurally. The cart 110, and subsystems thereof, may further comprise the ability to interface and communicate with one or more external imaging or image data management and communication systems, not limited to ultrasound, CT, fluoroscopy, cone beam CT, positron emission tomography (PET), PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri, intra and/or post histotripsy) and to provide access to and display of patient medical data including but not limited to laboratory and historical medical record data.

In some embodiments one or more carts may be configured to work together. As an example, one cart may comprise a bedside mobile cart equipped with one or more robotic arms enabled with a therapy transducer, and therapy generator/amplifier, etc., while a companion cart working in concert and at a distance of the patient may comprise integrated imaging and a console/display for controlling the robotic and therapy facets, analogous to a surgical robot and master/slave configurations.

FIG. 2 illustrates one embodiment of a histotripsy therapy and imaging system 200, including a coupling assembly 212. As described above, a histotripsy therapy and imaging system can include a therapy transducer 202, an imaging system 204, a robotic positioning arm 208, and a cart 210.

The therapy and/or imaging transducers are placed in the coupling assembly 212 which can further include a coupling membrane 214 and a membrane constraint 216 configured to prevent the membrane from expanding too far from the transducer. The coupling membrane 214 is filled with an acoustic coupling medium such as a fluid or a gel. The membrane constraint 216 can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 212 is supported by a mechanical support arm 218 which can be load bearing in the x-y plane but allow for manual or automated z-axis adjustment. The mechanical support arm 218 can be attached to the floor, the patient table, or the cart 210. The coupling assembly 212 is designed and configured to conform and hold the coupling membrane 214 in place against the patient's skin while still allowing movement of the therapy/imaging transducer 202, 204 relative to the patient the coupling membrane 214 with the robotic positioning arm 208.

The system can further include a fluidics system 220 that can include a fluid source, a cooling and degassing system, and a programmable control system. The fluidics system is configured for external loading of the coupling assembly 212 with automated control of fluidic sequences so that the coupling membrane 214 can conform around the patient.

### Histotripsy

Histotripsy is achieved by generating short, high amplitude, focused ultrasound pulses to generate a dense, energetic, “bubble cloud,” capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically affect tissue structure, and in some cases liquefy, suspend, solubilize and/or destruct tissue into sub-cellular components.

Histotripsy can be applied in various forms, including: 1) intrinsic-threshold histotripsy which delivers pulses typically with a 1-2 cycles of high amplitude negative/tensile phase pressure exceeding the intrinsic threshold to generate cavitation in the medium (e.g., ~24-28 MPa for water-based soft tissue), 2) shock-scattering histotripsy which delivers typically pulses 1-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) boiling histotripsy which employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous, and robotically mediated surgical procedures. It may be further targeted, planned, directed, and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism.”

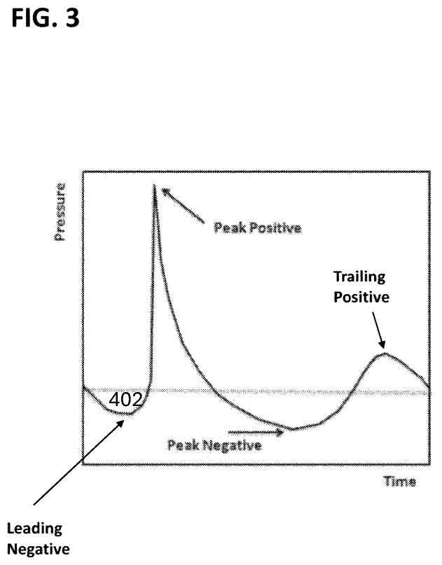

FIG. 3 illustrates an ultrasound pulse that can be used for shock scattering histotripsy. As shown the ultrasound pulse can include a leading negative half cycle, a peak positive half cycle, a peak negative half cycle, and a trailing peak positive half cycle (with the pulse traveling from right to left on the page). As shown, the trailing peak positive cycle has a lower amplitude than the peak positive cycle. This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface if the amplitude of those cycles is sufficient, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When the amplitude(s) of positive half cycle(s) of each pulse are limited, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism.”

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P-level for a region of interest (ROI), while the high-frequency pulse, which provides more precision, can pin-point a targeted location within the ROI and raise the peak negative pressure P-above the intrinsic threshold. This approach may be referred to as “dual frequency,” “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Bladder Therapy

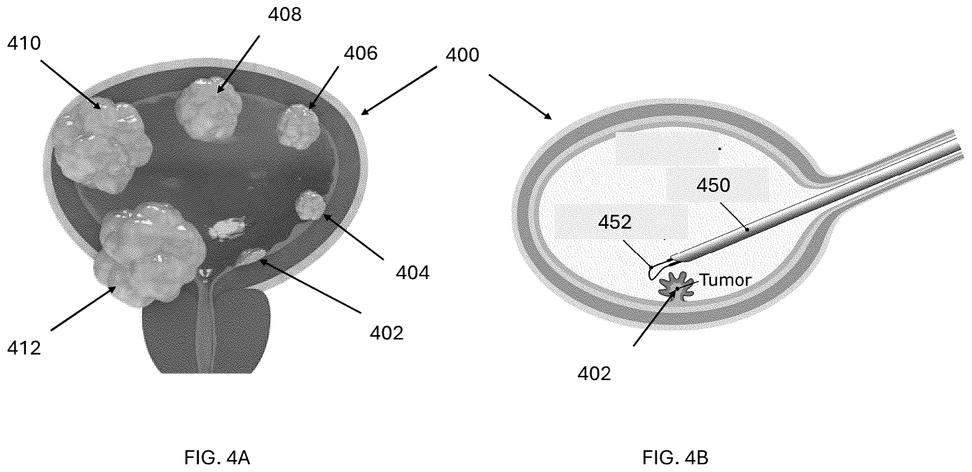

There are a variety of therapies available for the treatment of cancers of the bladder. The therapy pursued for a given patient depends in part on the size and extent of the tumor's invasion of the muscle layer of the bladder. Where the tumor is superficial, often referred to as a non-muscle invasive bladder cancer (NMIBC), and manifest just on the inner lining of the bladder a transurethral resection of the bladder tumor (TURBT) procedure is often employed. FIG. 4A depicts a graphic representation of a bladder 400, and tumors 402 and 404 which are NMIBC tumors. FIG. 4B depicts a resectoscope 450 inserted into the bladder 400. During a TURBT procedure the resectoscope 450 is transurethral inserted into the bladder. Viewing the tumor via the imager on the resectoscope, the superficial tumor 402 along with a portion the of the bladder lining and potentially a portion of the muscle layer is resected from the bladder (e.g., via radio frequency wire loop 452). This may be coupled with fulguration or laser therapy applied via tools inserted through the resectoscope 450 in an effort to ensure complete destruction of the cancerous tissue. In some instances, multiple TURBT procedures are performed (e.g., to ensure complete removal) for intermediate-and high-risk cancers.

Where the cancer is more extensive (e.g., a muscle-invasive bladder caner (MIBC)) a cystectomy may be performed. A partial cystectomy is performed where the tumor has invaded the muscle layer but is not very large and is only in once location on the bladder. As an example, tumors 406 and 408 in FIG. 4A may be amenable to treatment via a partial cystectomy. A partial cystectomy surgically removes the tumor (e.g., 406 and 408) and a portion of the wall of the bladder. After hole created as a result of the removed portion of the wall of the bladder is closed, for example, using stitches. Alternatively, a radical cystectomy may be undertaken where there are multiple tumors or the cancer is larger, and potentially even extending through the wall of the bladder, examples include tumors 410 and 412 in FIG. 4A. In a radical cystectomy removes the entire bladder from the patient. Cystectomies can be performed as either an open surgical procedure or a laparoscopic surgical procedure.

In addition to the surgical approaches, immunotherapies and chemotherapies can also be employed to treat the tumors. Some of the immuno-and chemo-therapies are prophylactic and applied following a TURBT procedure or partial cystectomy. In other instances, where one is seeking to avoid a radical cystectomy, these therapies may be used to reduce the size of the tumors (e.g., debulking) and potentially allow for the performance of a TURBT or partial cystectomy procedure. In some instances, intravesical application of chemo-or immune-therapies directly into the bladder is undertaken to target the destruction of the cancer cells in the bladder.

In addition, or as an alternative to one or more of the above procedures, external beam radiation therapy may be pursued. Radiation is often pursued in combination with chemotherapy. Finally, there exist some targeted therapy drugs that which attach to or act in combination with certain proteins that are found in the tumor.

While these procedures can be effective in treating bladder tumors, improved systems that do not result in the drawbacks associated with these procedures are always desirable. As described above, histotripsy is a non-invasive ablation method that delivers focused, microsecond-length, high-pressure, ultrasound pulses that create cavitation microbubbles in the target tissues, with no need for supplementary external agents. The rapid expansion and collapse of cavitation microbubbles produces high strain that mechanically disrupts cell membranes and structures in the target tissue, resulting in a liquefied and soluble tissue homogenate. Thus, histotripsy therapy is configured to liquefy, lyse, and/or solubilize the target tissue. The mechanically disrupted and soluble acellular debris generated by histotripsy is systemically reabsorbed by the body. Accordingly, the application of histotripsy to bladder tumors can address many of the challenges and shortcomings of existing therapies.

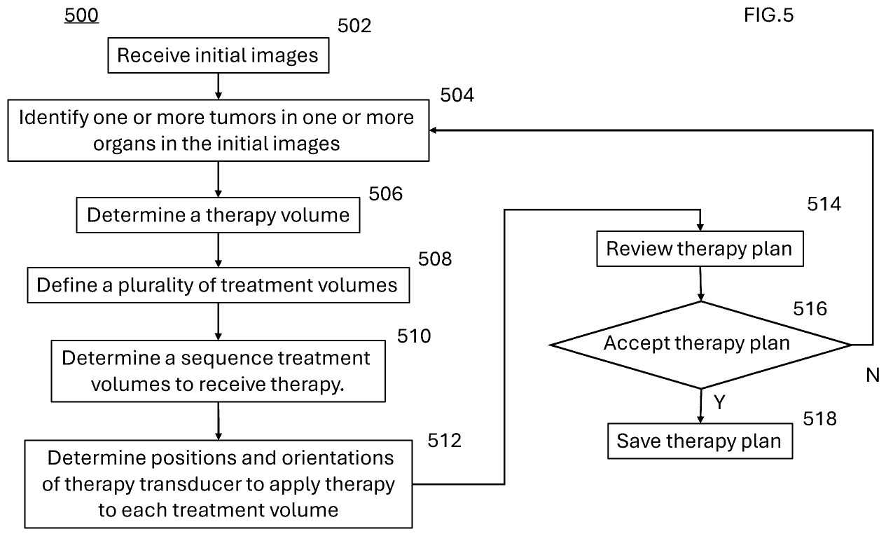

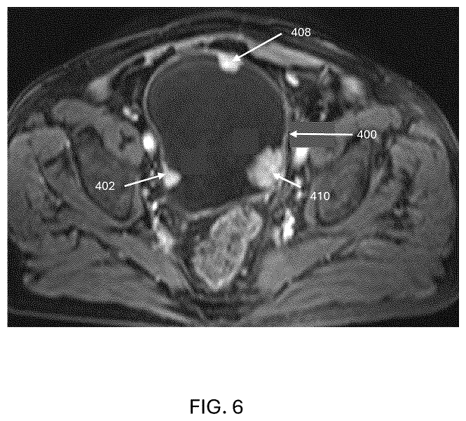

In accordance with the disclosure, FIG. 5 depicts a method 500 for treating bladder tumors using histotripsy. Like most histotripsy procedures, treatment of bladder tumors starts with the acquisition of images of the patient's bladder at step 502. These images may be ultrasound (e.g., 3D ultrasound), computed tomography (CT), magnetic resonance (MR) or from other imaging modalities without departing from the scope of the disclosure. FIG. 6 depicts an example of one image from a CT scan of a patient's bladder 400 depicting tumors 402, 408, and 410 manifest around the bladder 400. The acquired images are loaded into a planning application stored in a memory and executed by a processor in a computing device. The computing device may be on the cart 110 and associated with the control panel 106 or may be a separate computer such as a laptop or a tablet configured to receive the images. In some instances, the images may be stored in a hospital server system (e.g., an electronic medical record (EMR) system) or a cloud-based system accessible by the computing device.

At step 504 the images are displayed (e.g., on the control panel 106 or another display) and reviewed by the surgeon to identify the location of the tumor(s), the extent of the tumor's invasion into the muscle of the bladder, the size of the tumor and other factors. In addition to aspects of the tumor(s), various other features may be identified including the lining of the bladder, the muscle layer of the bladder and its thickness, the locations of the entrances to the bladder of the ureters, the location of the urethra, and the proximity of other structures of the patient's anatomy. The application allows for some or all of these features to be identified and/or marked in the images.

At step 506, using the features of the application, a therapy volume 602 (e.g., the volume of the tumor 404 and some margin 604) is defined. The therapy volume 602 will include at least a portion of the lining 414 of the bladder 400 and depending on the depth of invasion into the muscle layer 416 some portion of the muscle layer 416. When setting the margin 604, to maintain patency of the bladder 400, a residual muscle layer 606 must remain to avoid the need for surgical intervention to close any opening formed in the bladder 400. The residual muscle layer 606 (which in some instances may include some tumorous tissue) should be greater than a minimum thickness of muscle (and tumorous) tissue required to maintain patency and normal bladder functions.

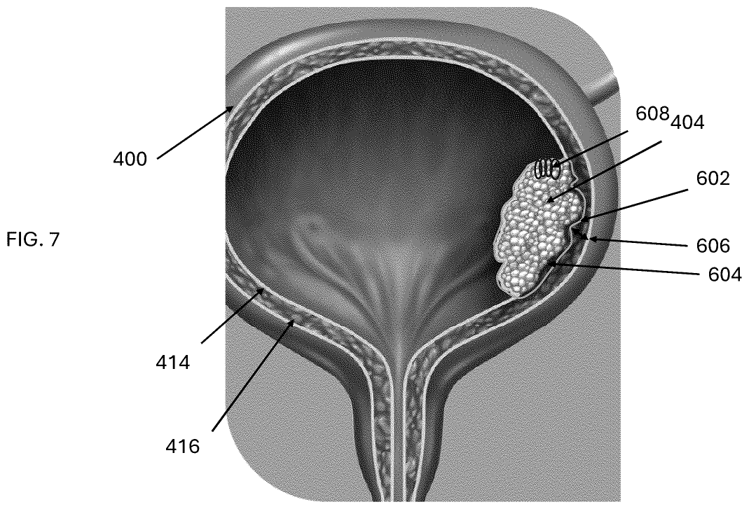

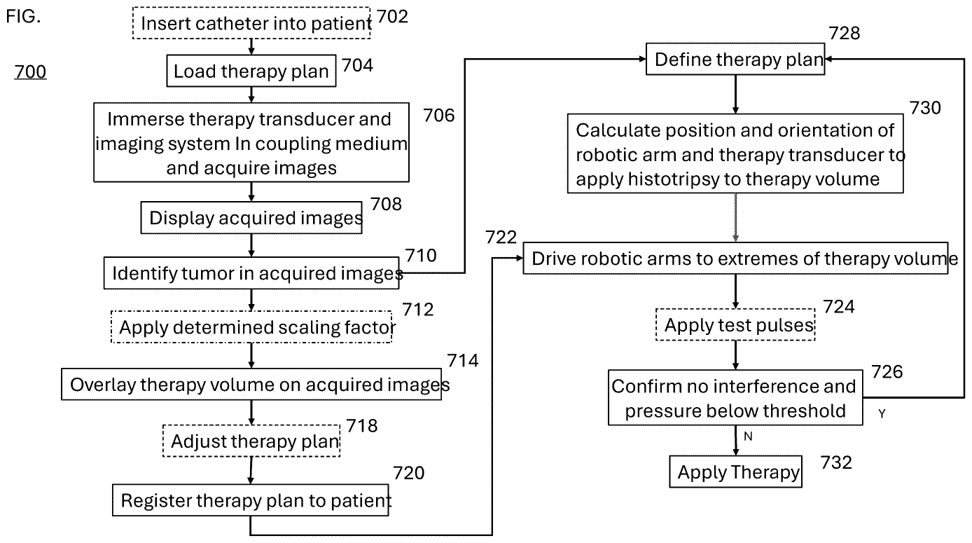

At step 508 a plurality of treatment volumes 608, within the therapy volume 602 are defined. As described above, the therapy transducer 102 emits an ultrasound signal to generate a volume of acoustic cavitation, a treatment volume 608, within which any tissue (both healthy and tumorous) is solubilized. The therapy volume 602 is defined by a plurality of treatment volumes 608. In some instances, the treatment volumes 608 overlap. Though depicted in FIG. 7 as having an ellipsoid shape, the treatment volume 608 can take other forms including round, cylindrical, and others without departing from the scope of the disclosure. Further, each individual treatment volume 608 may take on a different shape from neighboring treatment volume 608 to ensure complete treatment of the tumor and the desired margin 604. The shape of the individual treatment volumes 608 can be adjusted by changing the location of a focal point of the therapy transducer 102, the energy applied to portions of the therapy transducer 102 to achieve the desired shape of the treatment volume 608.

As will be appreciated, each treatment volume 608 is associated with a location and orientation of the robotic positioning arm 208 and therapy transducer 102 necessary to place the focal point of therapy transducer 102 substantially at a center of the treatment volume 608. At step 510, the application determines an efficient order for application of histotripsy to the plurality of treatment volumes 608. Associated with the order, at step 512 the application determines positions and orientations and the movements between such positions and orientations of the robotic positioning arm 208 and therapy transducer 102 to apply histotripsy therapy to every treatment volume 608 within the therapy volume 602. At step 514, the therapy plan, including the residual muscle layer 606, the therapy volume 602, the treatment volumes 608, the order of treatment volumes 608 to receive histotripsy, the proximity of critical structures (e.g., ureter or urethra) and other aspects can be reviewed by the surgeon. If at step 516 the therapy plan is rejected, the method returns to step 504 and following method steps described herein above, the therapy plan is adjusted as desired. If at step 516 the therapy plan is accepted the therapy plan is saved into a memory (e.g., a cloud storage, EMR, local memory, etc.) where the therapy plan can be accessed for execution, as described further below.

While described in connection with pre-procedure images, the disclosure is not so limited and the therapy plan, including the identification of the therapy volume 602, the residual muscle layer 606, the plurality of treatment volumes 608, the shape of the plurality of treatment volumes 608, and the order of applying histotripsy to the plurality of treatment volumes 608 can be generated using an application running on a computing device associated with the control panel 106 and cart 110. The therapy plan may be generated using ultrasound images acquired by the imaging system 204 with the patient positioned on the patient surface with the coupling assembly 212 positioned and filled with the coupling medium. Utilization of the imaging system 204, which is connected to the robotic positioning arm 208, is beneficial in that a location and orientation of the imaging system 204 when acquiring the images in the coordinate system of the robotic positioning arm 208 is known. As a result, the positions and orientations to which the robotic positioning arm 208 and therapy transducer 102 need to be navigated to apply histotripsy to each treatment volume 608 of the therapy volume 602 can be calculated from the acquired images.

To either generate a therapy plan or to begin a therapy, a patient must be placed on the patient surface and the coupling assembly 212 placed on the patient and filed with the coupling medium. Any air between the coupling assembly 212 and the patient is eliminated, and the therapy transducer 102 is lowered into the coupling assembly 212 and any air bubbles trapped under the therapy transducer 102 are removed. As a result of these steps, the therapy transducer 102 is acoustically coupled via the coupling medium to the patient such that ultrasound signals can pass from the therapy transducer 102 to a desired location within the patient (e.g., therapy volume 602).

Either following placement on the patient surface or as part of a pre-operative patient set up, a catheter (e.g., a foley catheter) is optionally inserted into the bladder 400 of the patient at step 702. Placement of a catheter is an optional step and may not be necessary based on the patient's anatomy and the surgeon's preferences. However, the catheter provides fluid communication and access to the bladder 400. The catheter may be connected to the fluidics system 220. By connection to the fluidics system 220, the volume of liquid within the bladder can be managed as desired by the surgeon. Adequate liquid within the bladder is necessary for acoustic coupling of the therapy transducer 102 to the tissues of the bladder. In addition, management of the fluid levels within the bladder allows for changes in shape and dimensions of the bladder. These changes in shape and volume can be observed in ultrasound images acquired, for example, via imaging system 104. By management of the volume of liquid within the bladder the disposition and positioning of the tumor may be altered. In addition, as described above, histotripsy occurs at the focal point of the therapy transducer 102. In some instances, it may be desirable to reduce the volume of liquid within the patient to ensure that the focal point can be navigated to a desired location within the bladder to achieve therapy. These and other operational considerations demonstrate the utility of the catheter during a histotripsy procedure on the bladder.

The catheter may also be employed to simply manage the volume of fluid within the bladder. Thus prior to the procedure the patient is prescribed ingestion of a desired volume of fluids, which over a specified period should be processed and rendered in the bladder. The catheter may be inserted into the bladder, and rather than inject additional fluids into the bladder, simply ensure that fluid does not escape the bladder to ensure acoustic coupling. A valve on the catheter can be utilized to selectively allow the flow of urine out of the bladder via the catheter to ensure a desired volume of water remains in the bladder during the histotripsy procedure while allowing excess volume to pass through the catheter.

In addition to the introduction of fluids to the bladder and the catheter may be configured to receive an endoscope. An endoscope inserted into the bladder enables visualization of the interior of the bladder and the application of the histotripsy therapy. As a result, in addition to ultrasound imaging of the application of the therapy (e.g., via imaging system 104) video (e.g., white light) imaging of the histotripsy procedure can be undertaken. The visualization of the procedure may be particularly beneficial when treating larger tumors where there is some risk of piercing the bladder wall during the therapy. The images captured by the endoscope enable the verification that the bladder remains patent throughout the procedure. Further, where a rupture or piercing of the bladder is observed, the surgeon can be alerted to the need for surgical intervention to close the opening in the bladder once created. As will be appreciated, in one or more aspects of the disclosure, the endoscope may be inserted into the bladder 400 without the catheter.

At step 704 a therapy plan is accessed by and loaded into a therapy application running on a computing device associated with the cart 110 and control panel 106. At step 706, with the therapy transducer 202 and the imaging system 204 immersed within the coupling medium in the coupling assembly 212, the robotic positioning arm 208 can be robotically or manually moved within the coupling assembly to acquire images of the tumor within bladder. At step 708, the therapy application displays the acquired images. During the histotripsy procedure the patient may be in a very different position and orientation during the procedure than they were during the pre-procedural images thus a plurality of images may be acquired at step 708, and optionally the acquired images (e.g., ultrasound images acquired by the imaging system 104) may be processed to form a 3D ultrasound image. Utilizing tools in the application, the boundary of the tumor identified in the therapy plan is identified in the acquired images at step 710. As will be appreciated, the identification of the tumor in the images may be manually performed (e.g., by the surgeon) or may be automatically performed by an image processing component of the therapy application. At step 712, with the boundaries of the tumor identified in the acquired images the application can optional determine whether and the magnitude of a scaling factor to apply to the therapy plan. The scaling factor adjusts the scale of the therapy plan to the scale of the images acquired by the imaging system 104, in this manner the surgeon can be confident that only the therapy volume 602 receives the histotripsy therapy.

At step 714, the therapy plan, and particularly the therapy volume 602 is overlaid on the images acquired by the imaging system 104. Because the position and orientation of the robotic positioning arm 208 and therapy transducer 102 when each of the images acquired by the imaging system is known, by overlaying the therapy volume 602 onto the tumor as it appears in the images, the therapy plan can be registered to the patient. In some instances, for a given image acquired by the imaging system 104 one or more tools in the application allow for the therapy volume to be rotated about one or more axes to closely match the plane in which the image is acquired. In some instances, multiple images (e.g., from orthogonal planes) may be matched to the therapy volume 602.

Optionally at step 718, the planned therapy volume 602 can be adjusted to account for changes of the margin 604, or the residual muscle layer 606. These changes may be caused by a variety of factors including the volume of fluid in bladder, increases in size of the tumor since the pre-procedure imaging, and others.

Once sufficient images from the imaging system 104 and the tumor identified therein are matched to the therapy volume 602, the therapy plan is registered to the patient at step 720. With the therapy plan registered to the patient, the robotic positioning arm 208 can then be driven to a plurality of positions at the extreme ends of the therapy volume 602 (e.g., extreme ends of X, Y, and Z axes of the therapy volume 602) at step 722. These extreme ends define the geometric extremes of the therapy volume 602, and where the robotic positioning arm 208 and the therapy transducer can be driven to these locations without interfering with the coupling assembly 212, or placing pressure on the patient in excess of a threshold, the surgeon can have confidence that the therapy plan can executed as planned.

As the therapy transducer 102 arrives at each one of the extremes of the therapy volume 602 a test pulse of histotripsy therapy can be optionally applied to the tumor at step 724. The test pulse can be observed in images acquired by the imaging system 104, and with the therapy plan overlaid on the tumor in the images. The histotripsy therapy is visible in the images and the presence of the histotripsy at the extremes of the therapy volume 602 can be either manually or automatically confirmed via the application. In addition, at step 726 the application confirms that there is no interference (impact of the therapy transducer 202 on the coupling assembly 212) and pressure applied to the patient is below a threshold.

In instances where there is no therapy plan to load into the application on the computing device associated with the cart 110 and the control panel 106 skips step 704 and following capture of images at step 706, and identification of the tumor in the acquire images a treatment plan can be defined on the acquired images at step 728. Definition of the treatment plan at step 728 including definition of the therapy volume 602, margin 604, residual muscle layer 606, and the size, shape, and locations of treatment volumes 608. As will be appreciated these features of the therapy plan may be defined in a number of images and images acquired along multiple axes. Further, the application may acquire multiple images along multiple axes and generate a 3D ultrasound image on which to identify the factors of the therapy plan described above.

Unlike using the pre-procedural images, by using the imaging system 104 which is connected to the robotic positioning arm 208, the position and orientation of the robotic positioning arm 208 and the imaging system 104 when each image is acquired is known in the robotic positioning arm coordinate system. As such the application running on the computing device is able at step 730 to calculate a positioning and orientation at which the therapy transducer 102 must be located to apply histotripsy therapy to each one of the treatment volumes 608 of the therapy volume 602. No separate registration step is required and the method can proceed to step 722 where the extremes of the therapy volume 602 to ensure that the therapy transducer 102 can be positioned and oriented at the extremes of therapy volume 602, as described above, without impacting the coupling assembly and without putting pressure on the patient in excess of a threshold.

If either the therapy transducer 102 impacts the coupling assembly 212 or the pressure applied to the patient by therapy transducer 102 on the patient exceeds a threshold, the method returns to step 728 and the therapy plan can be adjusted to avoid the impact or decrease the pressure applied to the patient. If the issue persists, the method ends and the coupling assembly 212 may be required to be moved on the patient, and the method 700 must be restarted.

Where no impact is detected and the pressure applied to the patient does not exceed a threshold, the therapy plan can be executed and histotripsy applied to each of the treatment volumes 608 that define the treatment volume 602 at step 732.

As noted above, the therapy plan and the therapy volume 602 need not apply histotripsy therapy to the entirety of a tumor. As an example, where the tumor is quite large, the therapy volume 602 may define less than the entirety of the tumor, and the application histotripsy can be utilized as a debulking process. During a debulking process some portion of the tumor is destroyed, but a portion remains. The debulking process may be part of a series of histotripsy procedures applied to the tumor to reduce the size of the tumor and enable one or more of the surgical procedures described above such as the minimally invasive laparoscopic partial cystectomy.

Still further, the application of histotripsy is associated with an immune response. The destruction of the cancerous results in the generation of T-cells and other immunogenic cells that attack any remaining cancer cells. This can be an entirely natural bodily response to the destruction of the cancerous cells and the resorption of the lysate generated by the treatment of the tumor. Alternatively, the lysate (liquified tissue) resulting from the application of the histotripsy therapy can be harvested (e.g., via the catheter), cultured, and reapplied to the patient, either locally at the tumor site, or systemically to trigger the immune response within the patient and the generation of the T-cells and other cancer cell antagonists which continue the application of the destruction of the cancer cells. The application of these immune response inducing may be repeated periodically after the histotripsy therapy. The immune response may be measured by a change in blood level data (e.g., number of T-cells in sample) of a sample prior to therapy and some period or multiple periods after therapy.

Still further, as part of the immune response, where the patient exhibits cancerous tumors in other organs or interstitial tissues, one or more of these tumors may be targeted for receiving histotripsy therapy in addition to the bladder tumor. In one aspect, this additional tumor is one that can be treated without requiring adjustment of the acoustic coupling assembly 212, thus avoiding many of the steps of method 700, described above, to achieve application of therapy to the tumor outside of the bladder.

As described herein, a catheter may be employed to ensure that sufficient liquid is present within the bladder. The liquid enables acoustic coupling to the tissues of the bladder. The catheter may be connected to the fluidics system 220 to allow for controlled filling of the bladder. Alternatively, where the patient protocol demands ingestion of a volume of fluid prior to the procedure, the catheter can include a valve which allows for control of the volume with the bladder and where necessary controlled release of the volume of liquid.

As will be appreciated, the bladder is a highly flexible organ. In addition to enabling acoustic coupling, the liquid within the bladder fills the volume of the bladder and separates the walls of the bladder from one another. Accordingly, management of the volume of liquid within the bladder enables separation of tissues to receive histotripsy therapy from those that are not to receive treatment. Accordingly, management of the volume of liquid within the bladder helps ensure effective targeting of tissue to receive therapy. One of skill in the art will recognize that ensuring sufficient liquid within the bladder also enhances the ability to image the bladder and the tumors therein via ultrasound.

Another aspect of the management of the liquid within the bladder is that the volume of fluid within the bladder can effect the distance between opposing sides of the bladder. Given its location within the patient, the bladder is beneficially approached from the anterior side of the patient, and thus there are generally few issues with hard tissues (e.g., bones) limiting access to the tumor. However, if the bladder is filled with a sufficient volume, the focal point of the therapy transducer 102 may not reach portions of the tumor. Thus, for example at step 722 as the robotic positioning arm 208 and therapy transducer are navigated to the extremes of the therapy volume 602, and optionally test pulses are applied at step 724 a determination may be made whether the focal point can be navigated to the extremes of the therapy volume 602. If the focal point cannot arrive at these extremes, some of the liquid within the bladder may be released from the bladder. The result is that the distance between the anterior portion of the bladder and the posterior portion of the bladder will be reduced and enable the focal point to be navigated deeper within the patient (e.g., when measured from the anterior of the patient).

Though generally described herein in terms of volume of liquid within the bladder, the disclosure is not so limited and instead a pressure of the liquid applied to the wall of the bladder can be managed. For example, the catheter may include a pressure relief valve that automatically allows liquid to drain from the bladder when sufficient pressure is achieved. This may be particularly useful when the patient ingests liquid prior to the procedure, and the ureters will be draining urine from the kidneys to the bladder throughout the duration of the procedure. Management of the pressure within the bladder may beneficially ensure that the residual muscle layer 606 is not overstressed during the procedure.

In conjunction with the pressure relief valve, the bladder may in fact be over filled (e.g., to 100+percentage) of its capacity. Filling of the bladder may in some instances beneficially present the bladder in proximity to the abdominal wall and thus enhance the acoustic coupling or lessen the degradation of the acoustic pathway as the ultrasound signals traverse the tissues of the patient.

Described herein above is the concept of the use of an endoscope to visualize the tumors within the bladder. In a further aspect of the disclosure, the endoscope is a rigid endoscope. The endoscope may include a localization component (e.g., EM sensor, optical sensor, etc.). A similar sensor may be placed on the therapy transducer 102. With the endoscope visualizing the tumors from within the patient, a relative position determination between the imaging component of the endoscope and the therapy transducer 102 can be made by the therapy application. This relative position may beneficially enable the tracking in real time of the tumor and the therapy transducer 102 during the application of the therapy. The endoscope may include a range finder to map the position of the bladder wall and the tumor to enhance registration of the therapy plan to the tumor as it sits in vivo. The endoscope may also include functionality to irrigate and flush the bladder and the therapy site of the destroyed tissue.

Yet a further aspect of the disclosure relates to the use of one or more mesh products to promote security and regrowth of the tissues of the bladder following therapy. There have been developed a number of repair meshes employed to repair perforated tissues. These meshes adhere to the tissue with the perforation. The mesh works as a lattice structure through which new tissue regrows. The mesh itself over the course of weeks or months is resorbed by the body. Use of such a mesh, deployed via the endoscope can allow a residual muscle layer 606 to be further reduced and allow for greater tumor destruction. Post therapy application of the mesh mechanically supports the residual tissue layer 606 until such time as sufficient tissue has regrown.

Further, the mesh products may be employed in situations where a perforation develops in the bladder during the application of therapy. The mesh product acts as a temporary seal to the bladder allowing normal or substantially normal organ function and may be deployed and positioned without requiring more invasive procedures (e.g., laparoscopic surgical procedures) to repair the bladder. The mesh product may be coated with adhesion promoting features including barbs and the like, and may also be coated with therapeutic agents, antibiotic agents, immune response enhancing agents, and others.

Described herein are applications operable on one or more computing devices. Those of skill in the art will recognize that the three applications may be modules of a single histotripsy application and that the applications share features which allow aspects of one module to connect to another module without departing from the scope of the disclosure.

### EXAMPLES

### Example 1

The embodiments described above are cited by way of example, and that the present invention is not limited to what has been particularly shown and described hereinabove. Rather, the scope of this disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are considered an integral part of the application.

We claim:

1. A system for applying histotripsy therapy, comprising:
a catheter insertable through a body lumen to fluidly connect a bladder of a patient;
an acoustic coupling assembly configured for placement on the patient;
a fluid source and configured to supply an acoustic coupling fluid acoustic coupling assembly; and
a therapy transducer configured to emit an ultrasound signal and generate a bubble cloud at a focal point of the therapy transducer, wherein the therapy transducer is configured to acoustically couple to tissues of the bladder of the patient via the acoustic coupling assembly and fluid within the bladder of the patient.

2. The system of claim 1, wherein the acoustic coupling assembly comprises a frame having two openings, wherein a first opening is configured to receive and mount an elastic membrane, and a second opening is configured to receive acoustic coupling fluid and the therapy transducer.

3. The system of claim 2, wherein the acoustic coupling fluid is degassed water.

4. The system of claim 2 further comprising a robotic arm coupled to the therapy transducer.

5. The system of claim 4, further comprising an imaging system configured to emit and receive an ultrasound signal for generation of images.

6. The system of claim 5, further comprising a computing device including a memory and a processor, the memory storing instructions that when executed by the processor cause the processor to:
generate and display an image from signals received by the imaging system.

7. The system of claim 6, wherein the image depicts a target for application of therapy.

8. The system of claim 7, wherein the memory stores therein a therapy plan for applying histotripsy to a therapy volume.

9. The system of claim 8, wherein the therapy plan is developed from pre-procedure images of the patient's bladder.

10. The system of claim 9, wherein the pre-procedure images are selected from the group consisting of computed tomography, magnetic resonance, positron emissions tomography, optical coherence tomography, and ultrasound images.

11. The system of claim 8, wherein the memory stores therein instructions that when executed by the processor cause the robotic arm to perform a series of movements to confirm no interference between the robotic arm and the coupling assembly.

12. The system of claim 11, wherein the therapy plan defines a series of overlapping treatment volumes centered at focal points of the therapy transducer and a series of movements for the robotic arm to move to a position and orientation to generate a bubble cloud at each of the series of treatment volumes.

13. The system of claim 12, wherein the therapy plan defines a residual muscle layer of the bladder to retain patency of the bladder.

14. The system of claim 13, wherein the therapy plan defines a therapy volume such that less than an entirety of the target receives the therapy.

15. The system of claim 13, wherein the therapy plan applies therapy to an entirety of the target.

16. The system of claim 13, further comprising a display depicting the therapy plan overlaid on the image from signals received by the imaging system.

17. The system of claim 16, wherein the instructions when executed by the processor receives signals via the imaging system to generate images as the therapy is applied to target.

18. The system of claim 17, further comprising a pressure sensor configured to detect pressure applied to the patient by the therapy transducer.

19. The system of claim 18, further comprising a pressure sensor formed on the catheter and configured to detect a pressure of the acoustic coupling medium within the bladder of the patient.

20. The system of claim 19, further comprising a vacuum source in communication with the catheter and configured to remove the acoustic coupling medium from the bladder of the patient.
